(19) United States
(12) Patent Application Publication
(10) Pub. No.: US 2008/0317310 A1
(43) Pub. Date: Dec. 25, 2008

(54) Method and system for image processing and assessment of blockages of heart blood vessels

(76) Inventors: Mitta Suresh, Richardson, TX (US); Jude Dalton, Nottingham (GB)

Correspondence Address: MEYERTONS, HOOD, KIVLIN, KOWERT & GOETZEL, P.C., P.O. BOX 398, AUSTIN, TX, 78767-0398, US

(21) Appl. No.: 11/953,649

(22) Filed: Dec. 10, 2007

Related U.S. Application Data
(60) Provisional application No. 60/873,769, filed on Dec. 8, 2006.

(51) Int. Cl. A61B 5/00

(52) U.S. Cl. 382/130

(57) ABSTRACT

One embodiment discloses a computerized method of assessing deposits and/or blockages in blood vessels in a human, specifically in a human heart. The method may include inputting patient data and creating a computerized interactive model of a heart based on the patient data. Patient data may include a plurality of images of a least a portion of a human heart. Images may include three-dimensional images. An image may be divided into regions. A property of a region may be assessed. A property may include intensity of brightness of a region or a portion of a region. A region may include one or more voxels or one or more pixels. A method may include comparing a property of a region of a heart from a first image to a second image. The first image and the second image may include equivalent regions acquired during different time periods.

### PRIORITY CLAIM

This application claims priority to U.S. Provisional Patent Application No. 60/873,769 entitled “METHOD AND SYSTEM FOR IMAGE PROCESSING AND ASSESSMENT OF BLOCKAGES OF HEART BLOOD VESSELS” filed on Dec. 8, 2006, which is incorporated by reference herein.

### BACKGROUND OF THE INVENTION

1. Field of the Invention

This invention relates generally to methods and systems for assessing blockages in tubular structures or lumens, and in particular to a computerized system and method for assessing blockages in blood vessels in cardiac tissue of a human heart.

2. Description of the Related Art

The circulatory system of a human works as a closed system where the effects of one part of the system are felt by all other parts of the system. For example, if a person's blood pressure rises then there is a corresponding pressure decrease in the venous system, the decrease is much smaller than the increase in the arterial side because of the fact that venous vasculature is more compliant than the arterial vasculature. Within the circulatory system the key component is the heart. Any change to any component of the heart will have an effect felt throughout the entire system.

The primary function of a heart in an animal is to deliver life-supporting oxygenated blood to tissue throughout the body. This function is accomplished in four stages, each relating to a particular chamber of the heart. Initially, deoxygenated blood is received in the right auricle of the heart. This deoxygenated blood is pumped by the right ventricle of the heart to the lungs where the blood is oxygenated. The oxygenated blood is initially received in the left auricle of the heart and ultimately pumped by the left ventricle of the heart throughout the body. The left ventricular chamber of the heart is of particular importance in this process as it is responsible for pumping the oxygenated blood through the aortic valve and ultimately throughout the entire vascular system.

Coronary heart diseases are one of the main causes of death in the industrialized world. They are often triggered by atherosclerotic plaque which gathers in the coronary vessels and which can lead to narrowing or occlusion of the vessels. Atherosclerotic plaque can be divided into various types with different compositions.

Lipid-rich or noncalcified plaque, also referred to as soft plaque, is associated with a particularly high risk of a coronary event such as an infarct or sudden cardiac death, because its rupture most likely leads to an acute vascular occlusion. In cases where soft plaque is present, the risk of an acute coronary event can be reduced by using certain medicines called lipid-lowering agents. In contrast to soft plaque, another type of plaque called calcified plaque more rarely causes acute vascular occlusions. The same applies to fibrous plaque, an intermediate stage between soft plaque and calcified plaque.

When using imaging techniques, it is therefore of advantage to be able to detect the presence of soft plaque in the patient's coronary vessels as quickly as possible. Known imaging methods for visualizing soft plaque in coronary vessels are the invasive imaging methods of intravascular ultrasound imaging (IVUS) or optical coherence tomography (OCT). These imaging techniques generate gray-scale images whose image plane is oriented perpendicular to the vessel axis. The vessel can be seen as a concentric ring in the center of the image, and different plaque types can be pinpointed by different gray-scale scale areas in the image. However, the observer must have considerable experience to reliably detect the presence of plaque and to be able to differentiate between the different types of plaque.

Since the introduction of multi-slice computed tomography machines, which can record four or more slices simultaneously by way of a suitable detector array, noninvasive imaging of the heart is also possible in conjunction with electrocardiographically synchronized operation (ECG gating). ECG gating, in conjunction with the high recording speed of a multi-slice computed tomography machine, permits visualization of the coronary arteries with minimal movement artifacts. The recorded two-dimensional slice images can then be visualized in different ways, for example by three-dimensional volume rendering (VRT) or by two-dimensional thin-slice MIP (maximal intensity projection).

However, when viewing the two-dimensional slice images of the examined area which have been obtained with the imaging tomographic technique, a problem which often arises is that of the poor level of detection of the different types of plaque in relation to the surrounding tissue.

Even with the aid of the currently available imaging techniques, it is a time-consuming and complex process to evaluate the coronary vessel system, for example in order to measure stenoses or to estimate the extent of calcified or non-calcified plaque deposits. Different visualization methods with the aid of which the recorded vessel structures can be displayed are made available with the aid of the high computing ability of modern image computers. Examples of this are MIP (Maximum Intensity Projection), VRT (Volume Rendering Technique), SSD (Shadow Surface Display) or else combinations of these visualization methods that support the radiologist during diagnosis. A quantitative analysis of the vessel structures requires a segmentation of the structures from the two-dimensional or three-dimensional recorded images on the basis of which it is possible to measure quantitative variables such as, for example, the length or the diameter/length ratio of a stenosis.

Relaying the recorded data or the data derived from the recorded images to other specialists, for example a cardiologist, constitutes a particular problem. The visualization methods used to date such as, for example, interactive three-dimensional-VRT leads to images that are difficult to interpret in the context of a reduction to a two-dimensional display.

Despite the state of digitization techniques and electronic networking in hospitals, printing such images out onto paper is frequently still always required in order to transmit the examination results to appropriate specialists for providing a diagnosis. In these instances, the investigation result is therefore generally accompanied by a report in which the vessel tree is described in simple words, for example by specifying the distance of a lesion from a fixed landmark such as, for example, a branch point or an anatomical abnormality. However, even with an accompanying report, it is frequently difficult for the person skilled in the art to reconstruct the actual vessel structure correctly from the two-dimensional images.

Various treatments are currently employed to repair, replace or mitigate the effects of damaged components of the heart. Some of these treatments involve grafting new arteries onto blocked arteries, repairing or replacing valves, reconstructing a dilated left ventricle, administering medication, or implanting mechanical devices. All these treatments apply standard repairs to unique problems with a minimum of analysis as to what the optimum intervention should involve. Typically, the current procedures do not involve analyzing the performance of the cardiac system after the treatment to see what effect the treatment has had on the entire system. For example, a patient with blocked arteries may undergo a standard treatment of placing 5-6 grafts on their heart due solely to a short visual inspection of angiographic films that show some stenosis of the arteries of the heart. No analysis is performed to see if placing 3-4 grafts will achieve the same perfusion of the myocardium as the 5-6 grafts. It is simply a situation where the user decides that more is better, which may not be true. Placing 5-6 grafts requires more surgical time, longer pump runs, and incisions into numerous areas of the body to recover the needed grafts. This increases morbidity to the patient and may contribute to death of the patient who may not tolerate the additional stress of a longer, more invasive procedure. On some patients, the extra grafts may be needed, since collateral flow, or flow from other arteries, is not sufficient to perfuse the entire myocardium. On other patients, the grafts may not be needed, since sufficient flows will be generated from fewer grafts. Currently, the user has no way of knowing if the total number of grafts that he put in was appropriate.

A similar procedure is used to place stents in a vessel. Stents are placed in vessels based on an assessment of blockage and ability to access the obstructed area. No method of analysis is performed to determine the effects of placing a stent, to analyze how many stents should be placed, and/or to determine if the placement of stents produces a better result than bypassing.

What is needed, therefore, is a reliable method and apparatus to allow a user to assess blockages in, for example, blood vessels in the heart. It is also desirable to have a method and apparatus for assessing blockages which at least a portion of is automated. A user may simply initiate a process which finds and indicates blockages using provided images of a subject's heart.

### SUMMARY

In some embodiments, a method may include imaging blood vessels in a human body. A method may include providing a plurality of three-dimensional images of at least a portion of a human body acquired over a period of time to a computer system. The plurality of images may include at least a first image and a second image acquired at different times. The method may include dividing the first image and the second image into a plurality of regions. Each of the regions may correspond between the first image and the second image. A method may include assessing a property in a plurality of regions of the body from the first image. A method may include assessing the property in a corresponding region of the body from the second image.

In some embodiments, a method may include comparing the property of the regions of the body from the first image to the property of the regions of the body from the second image to select either a region from the first image or a corresponding region from the second image. A method may include creating a third image of at least a portion of human blood vessels using the selected regions.

In some embodiments, a first image and a second image may include at least a portion of a human heart.

In some embodiments, a region comprises one or more voxels.

In some embodiments, a property comprises an intensity of a region. Comparing the property of the regions may include using a mathematical operator to compare the regions. The mathematical operator may include the operator greater than.

In some embodiments, a method may include creating a third image of blood vessels of the body using the selected regions. The third image may at least appear three-dimensional. The third image may be two-dimensional.

In some embodiments, at least a portion of a plurality of three-dimensional images may be acquired using computed tomography imaging and/or magnetic resonance imaging.

In some embodiments, a method may include assessing blockages in blood vessels in a human body. A method may include providing at least one three-dimensional image of at least a portion of a human body to a computer system. A method may include virtually positioning a cell in a blood vessel depicted in at least one of the images of the body. A method may include virtually moving the cell through the blood vessel such that a volume of the cell remains constant. A method may include assessing positions along the blood vessel the cell changes from a first shape to a second shape.

In some embodiments, a method may include providing at least two three-dimensional images of at least a portion of a human body. At least a first image and a second image may be acquired at different times.

In some embodiments, a method may include assessing changes of the cell's shape at corresponding positions in at least a second image acquired at a different time to the assessed positions. A method may include indicating positions in the blood vessel where the cell changes from a first shape to a second shape in at least the second image.

In some embodiments, a method may include creating an image depicting the assessed positions along the blood vessel where the cell changes from a first shape to a second shape. The created image may be two-dimensional. The created image may at least appear three-dimensional. The created image may at least appear four-dimensional.

In some embodiments, a method may include virtually moving the cell through the blood vessel. The blood vessel may be defined by a predetermined pixel intensity range. The cell may be defined by a number of voxels. At least one of the provided three-dimensional images may include at least a portion of a human heart.

In some embodiments, at least a portion of a plurality of three-dimensional images may be acquired using computed tomography imaging and/or magnetic resonance imaging.

In some embodiments, a method may include facilitating transfer of data related to blockages in human body blood vessels. A method may include providing one or more three-dimensional images of at least a portion of a human body to a computer system. A method may include assessing blockages in blood vessels in a human body using one or more of the images. A method may include creating an image of at least a portion of a human body indicating the assessed blockages. A method may include reducing the resolution of portions of the created image outside of regions of the created image comprising the assessed blockages such that a size of the data package forming the created image is reduced.

One or more of the provided three-dimensional images may include at least a portion of a human heart.

In some embodiments, a method may include providing a plurality of three-dimensional images of at least a portion of a human body acquired over a period of time.

A created image may at least appear four-dimensional. A created image may at least appear three-dimensional. A created image may be two-dimensional.

In some embodiments, at least a portion of a plurality of three-dimensional images may be acquired using computed tomography imaging and/or magnetic resonance imaging.

In some embodiments, a method may include creating a new three-dimensional data set from a series of different three-dimensional datasets. This may be accomplished by selecting a particular region of interest from each of the different data sets. This may assist in reducing the file size to be transferred since important information from multiple phases is combined into single phase. For example, certain coronary arteries such as LAD typically appears best at 70% RR phase, while RCA, another coronary artery, typically appears best in 30% RR phase. A new three-dimensional data set may be created where the pixel data in the vicinity of LAD area is taken from the 70% RR phase while the area around RCA is taken from the 30% phase.

In some embodiments, a method may include imaging calcium in blood vessels in a human heart. A method may include providing at least a first image of at least a portion of a human body to a computer system. A method may include assessing a position of blood vessels of a human heart within the first image by assessing an intensity in a plurality of voxels from the first image. A method may include providing at least a second image of at least a portion of the human body. A method may include assessing a position of the heart within the second image using the assessed position of blood vessels in the first image.

In some embodiments, a method may include assessing calcium within the blood vessels associated with the heart.

In some embodiments, a method may include creating a third image depicting calcium within the blood vessels of the heart from the second image. A created image may at least appear four-dimensional. A created image may at least appear three-dimensional. A created image may be two-dimensional.

In some embodiments, a first image and a second image may include at least a portion of a heart. A first image may be a three-dimensional C positive image. A second image may be a three-dimensional C negative image.

In some embodiments, a method may include assessing soft plaque in blood vessel walls in a human body.

In some embodiments, a method may include combining coronary images and viability images. A method may include providing at least one coronary image of at least a portion of a human body to a computer system. A method may include providing at least one viability image of at least a portion of a human body to a computer system. A method may include combining at least one of the coronary images with at least one of the viability images using at least one feature to spatially align the images.

In some embodiments, at least one of the coronary images and/or at least one of the viability images includes at least a portion of a heart.

In some embodiments, at least one of the coronary images and/or at least one of the viability images at least appears three-dimensional or at least appears four-dimensional.

In some embodiments, at least one of the features is an anatomical landmark. The anatomical landmark may include at least a portion of a spine. The anatomical landmark may include at least a portion of a rib.

In some embodiments, a method may include creating an image comprising at least some of the features depicted in at least one of the coronary images and at least one of the viability images.

In some embodiments, a method may include creating an image comprising at least some of the features depicted in at least one of the coronary images and at least one of the viability images. A created image may at least appear four-dimensional. A created image may at least appear three-dimensional. A created image may be two-dimensional.

In some embodiments, a method may include assessing a state of a human heart. A method may include providing one or more viability images of at least a portion of a human heart to a computer system. A method may include calculating a quantitative metric using one or more features derived from one or more of the viability images of the human heart. A method may include assessing a state of the human heart using the quantitative metric.

In some embodiments, at least one of the viability images may be acquired using computed tomography imaging and/or magnetic resonance imaging.

In some embodiments, at least one of the features may be a size of an infarct. The size may include a mass of the infarct. The size may include an area of the infarct. The size may include a size of an infarct as a percentage of a ventricle size.

In some embodiments, at least one of the features may be an area of the infarct that is in contact with viable muscle.

In some embodiments, at least one feature may include to identifying no reflow areas within infarct areas. No reflow areas within infarct areas may be identified areas of hypoenhancement within the region of hyperenhancement. This is an indication of microvascular obstruction. No reflow or microvascular Obstruction (MVO) areas may be quantified as area, volume or mass. A new metric that is a function of one all of the following factors: infarct size, MVO, LV volumes, EF, transmurality of scar may help identify patients susceptible to heart failure. Since all the variables of the metric are available, the metric may be automatically calculated.

In some embodiments, at least one of the features may be a morphology of the infarct.

In some embodiments, at least one of the features may be a ratio of viable but akinetic muscle to non-viable muscle.

In some embodiments, a method may include assessing the heart's risk factor of Sudden Cardiac Death.

In some embodiments, a method may include assessing the heart's risk factor of V-tach.

In some embodiments, a method may include acquiring computed tomography images of a human body. A method may include administering a first dose of contrast agent to a human body. A method may include waiting a predetermined period of time. A method may include administering a second dose of contrast agent to the human body. A method may include acquiring at least one computed tomography image of at least a portion of the human body.

The predetermined period of time may range from about 5 to 10 minutes, 2 to 15 minutes, or 10 to 30 minutes. The first dose may at least partially deposit itself in one or more of the infarcted regions of the heart during the predetermined period of time. The second dose may at least effectively illuminate at least some of the coronaries. This multiple dose method may allow both coronaries and infarcted tissue are captured in the same acquisition.

At least one of the computed tomography images may include at least a portion of a heart. In some embodiments, a first dose and/or a second dose may be administered orally, subcutaneously, percutaneously, and/or intravenously.

In an embodiment a system may function to employ any of the methods described herein. The system may include a CPU. The system may include a system memory coupled to the CPU. The system memory may store one or more computer programs executable by the CPU. One or more computer programs may be executable to perform any of the methods outlined herein.

In some embodiments, a carrier medium may function to store program instructions. The prograin instructions may be executable to implement a method as described herein.

In an embodiment, a report may include a description of a result or an effect of a method as described herein.

In some embodiments, a method as described herein may include assessing a cost to be charged to a user for using the method based on a number of times the user applies the method.

### BRIEF DESCRIPTION OF THE DRAWINGS

Advantages of the present invention may become apparent to those skilled in the art with the benefit of the following detailed description of the preferred embodiments and upon reference to the accompanying drawings in which:

FIG. 1 depicts a network diagram of an embodiment of a wide area network that may be suitable for implementing various embodiments.

FIG. 2 depicts an illustration of an embodiment of a computer system that may be suitable for implementing various embodiments.

While the invention is susceptible to various modifications and alternative forms, specific embodiments thereof are shown by way of example in the drawings and may herein be described in detail. The drawings may not be to scale. It should be understood, however, that the drawings and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION

It is to be understood the present invention is not limited to particular devices or biological systems, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Thus, for example, reference to “a computer system” includes one or more computer systems.

### DEFINITIONS

Unless defined otherwise, all technical and scientific terms used herein have the same meaning as commonly understood by one of ordinary skill in the art.

The term “blockage,” as used herein, generally refers to obstructing something (e.g., a lumen) by placing obstacles (e.g., calcium) in the way, a lumen may not be totally obstructed, but may merely be restricted.

The term “contrast agent,” as used herein, generally refers to a “dye” used to highlight specific areas so that the organs, blood vessels, and/or tissues are more visible. By increasing the visibility of all surfaces of the organ or tissue being studied, they can help a radiologist determine the presence and extent of disease or injury

The term “corresponding,” as used herein, generally refers to having the same or nearly the same relationship (e.g., corresponding portions of two images of a ROI are images of the same or nearly the same segment of a human body).

The phrase “four-dimensional image,” as used herein, generally refers to exhibiting four dimensions, such as the three spatial dimensions and single temporal dimension of relativity theory. In some embodiments, a four-dimensional image may merely give the illusion of depth, but may in actuality consist of a two-dimensional image on, for example, a computer screen or a printed piece of paper.

The term “lumen,” as used herein, generally refers to an inner open space or cavity of a tubular organ (e.g., a blood vessel or an intestine).

The term “mathematical operator,” as used herein, generally refers to a symbol for expressing a mathematical operation, a function, esp. one transforming a function, set, etc., into another.

The term “metric” as used herein, generally refers to nonnegative real-valued function having properties analogous to those of the distance between points on a real line, as the distance between two points being independent of the order of the points, the distance between two points being zero if, and only if, the two points coincide, and the distance between two points being less than or equal to the sum of the distances from each point to an arbitrary third point.

The term “organ,” as used herein, when used in reference to a part of the body of an animal or of a human generally refers to the collection of cells, tissues, connective tissues, fluids and structures that are part of a structure in an animal or a human that is capable of performing some specialized physiological function. Groups of organs constitute one or more specialized body systems. The specialized function performed by an organ is typically essential to the life or to the overall well-being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, cardiovascular, lymphatic, immune, musculoskeletal, nervous, digestive, endocrine, exocrine, hepato-biliary, reproductive, and urinary systems. In animals, the organs are generally made up of several tissues, one of which usually predominates, and determines the principal function of the organ.

The terms “pharmaceutically or nutraceutically acceptable formulation,” as used herein, generally refers to a non-toxic formulation containing a predetermined dosage of a pharmaceutical and/or nutraceutical composition, wherein the dosage of the pharmaceutical and/or nutraceutical composition is adequate to achieve a desired biological outcome. The meaning of the term may generally include an appropriate delivery vehicle that is suitable for properly delivering the pharmaceutical composition in order to achieve the desired biological outcome.

The term “pharmacologically inert,” as used herein, generally refers to a compound, additive, binder, vehicle, and the like, that is substantially free of any pharmacologic or “drug-like” activity.

The term “pixel,” as used herein, generally refers to the basic unit of the composition of an image on a television screen, computer monitor, or similar display.

The terms “reducing,” “inhibiting” and “ameliorating,” as used herein, when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

The term “subject,” as used herein, may be generally defined as all mammals, in particular humans.

The phrase “therapeutically effective amount,” as used herein, generally refers to an amount of a drug or pharmaceutical composition that will elicit at least one desired biological or physiological response of a cell, a tissue, a system, animal or human that is being sought by a researcher, veterinarian, physician or other caregiver.

The phrase “three-dimensional image,” as used herein, generally refers to involving or relating to three dimensions or aspects. A three-dimensional image may merely give the illusion of depth, but may in actuality consist of a two-dimensional image on, for example, a computer screen or a printed piece of paper.

The term “tissue,” as used herein, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

The term “voxel,” as used herein, generally refers to the smallest distinguishable box-shaped part of a three-dimensional space. A particular voxel will be identified by the x, y and z coordinates of one of its eight corners, or perhaps its centre. The term is used in three-dimensional modeling.

Cardiovascular disease (CVD) is the leading cause of death in a number of different countries. This disease stems from the underlying problem of atherosclerosis, which is a build up of plaque (consisting of substances including, among others, cholesterol and calcium) on the interior surface of arteries supplying the heart. Coronary heart disease typically manifests in two forms: heart attack and angina. A heart attack occurs when blood flow is completely blocked, typically from a dislodged portion of plaque. Angina, typically brought on by physical activity, is a chest pain or discomfort caused by an inadequate blood flow due to the narrowed artery. Computed tomography angiography (CTA) has emerged as the imaging modality of choice for diagnosing and planning treatment for coronary heart disease. An intravenous contrast agent (e.g., iodine-based dye or another substance with high molecular weight) may be used to enhance the visibility of blood, and hence the carrier vessels. The areas containing the in vivo contrast agent are marked in the resultant output images with a large Hounsfield unit (HU). Radiologists and cardiac surgeons require tools to help identify and visualise stenosis within the coronary arteries. Current medical imaging workstations include a number of two-dimensional tools such as multiplanar reformatting (MPR), oblique sectioning, and maximum intensity projection (MIP). To help manage the three-dimensional information, state of the art workstations are now beginning to include surface rendering algorithms. Unfortunately, surface rendering approaches (whereby an explicit surface is extracted and converted to polygons by the Marching Cubes algorithm) typically suffer from the problem of information occlusion, in which external surfaces obstruct internal surfaces. While solving the issue of information occlusion, traditional direct volume rendering (whereby surfaces of interest are interactively classified using transfer functions) can suffer from the opposite problem of information overload. Information overload occurs when too many input pixels are mapped to a single output pixel, typically resulting in blurry images.

Methods and apparatus of various embodiments will be described generally with reference to the drawings for the purpose of illustrating the particular embodiments only, and not for purposes of limiting the same. The illustrated embodiments address the ability of a user (e.g., a physician) to accurately assess the effects of cardiac disease (e.g., blockage in a cardiac blood vessel) on an individual patient and to use an appropriate treatment to restore the cardiac system to its optimal or best acceptable condition. In one embodiment, this is accomplished by using an analytical tool that takes images of the patient's own heart and collects other data related to the functioning of the heart. The collected data may be used to create a multi-dimensional finite element model and/or image of the heart. The multi-dimensional finite element image of the patient's heart may interact and respond to other models or a set of models. For example, the model of the patient's heart may also be connected to a model of the circulatory system and/or a model of the cardiac system. These models, in combination, may simulate the performance of the heart and its effect on the circulatory system. The use of these models may allow a user to determine the appropriate areas of the heart to be repaired, replaced, or otherwise medically treated for the patient. The models may also allow the user to determine the effects that the treatment may have on the portions of the heart and/or on the entire heart.

In an embodiment, a cardiac intervention process may include diagnosis, designing and/or manufacturing cardiac instruments, creating a procedure for cardiac modification, and/or prescribing a treatment of a cardiac disease. A cardiac disease may include any cardiac irregularity. A cardiac irregularity may be associated with a structural defect or abnormality of a heart. Other cardiac irregularities may be associated with a chemical or hormonal imbalance. Additional cardiac irregularities may include electrical abnormalities (e.g., arrhythmia). A method may include analyzing and performing a virtual treatment of a cardiac irregularity. A method of performing a virtual cardiac intervention may be performed on a computer system. A computer system may be a local computer system, including, but not limited to, a personal computer. Other embodiments may include remote systems or two or more computers connected over a network.

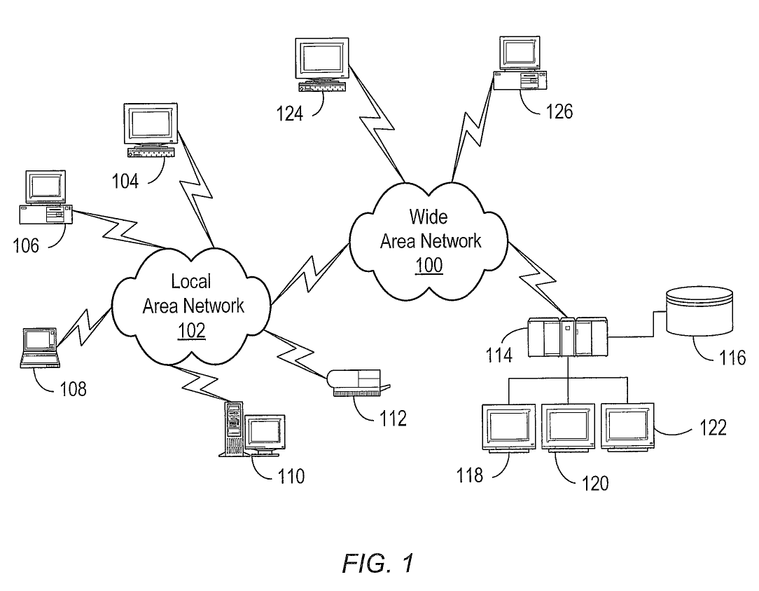

FIG. 1 illustrates a wide area network (“WAN”) according to one embodiment. WAN 100 may be a network that spans a relatively large geographical area. The Internet is an example of a WAN. WAN 100 typically includes a plurality of computer systems that may be interconnected through one or more networks. Although one particular configuration is shown in FIG. 1, WAN 100 may include a variety of heterogeneous computer systems and networks that may be interconnected in a variety of ways and that may run a variety of software applications.

One or more local area networks (“LANs”) 102 may be coupled to WAN 100. LAN 102 may be a network that spans a relatively small area. Typically, LAN 102 may be confined to a single building or group of buildings. Each node (i.e., individual computer system or device) on LAN 102 may have its own CPU with which it may execute programs, and each node may also be able to access data and devices anywhere on LAN 102. LAN 102, thus, may allow many users to share devices (e.g., printers) and data stored on file servers. LAN 102 may be characterized by a variety of types of topology (i.e., the geometric arrangement of devices on the network), of protocols (i.e., the rules and encoding specifications for sending data and whether the network uses a peer-to-peer or client/server architecture), and of media (e.g., twisted-pair wire, coaxial cables, fiber optic cables, and/or radio waves).

Each LAN 102 may include a plurality of interconnected computer systems and optionally one or more other devices such as one or more workstations 104, one or more personal computers 106, one or more laptop or notebook computer systems 108, one or more server computer systems 110, and one or more network printers 112. As illustrated in FIG. 1, an example of LAN 102 may include at least one of each of computer systems 104, 106, 108, and 110, and at least one printer 112. LAN 102 may be coupled to other computer systems and/or other devices and/or other LANs 102 through WAN 100.

One or more mainframe computer systems 114 may be coupled to WAN 100. As shown, mainframe 114 may be coupled to a storage device or file server 116 and mainframe terminals 118, 120, and 122. Mainframe terminals 118, 120, and 122 may access data stored in the storage device or file server 116 coupled to or included in mainframe computer system 114.

WAN 100 may also include computer systems connected to WAN 100 individually and not through LAN 102 such as, for purposes of example, workstation 124 and personal computer 126. For example, WAN 100 may include computer systems that may be geographically remote and connected to each other through the Internet.

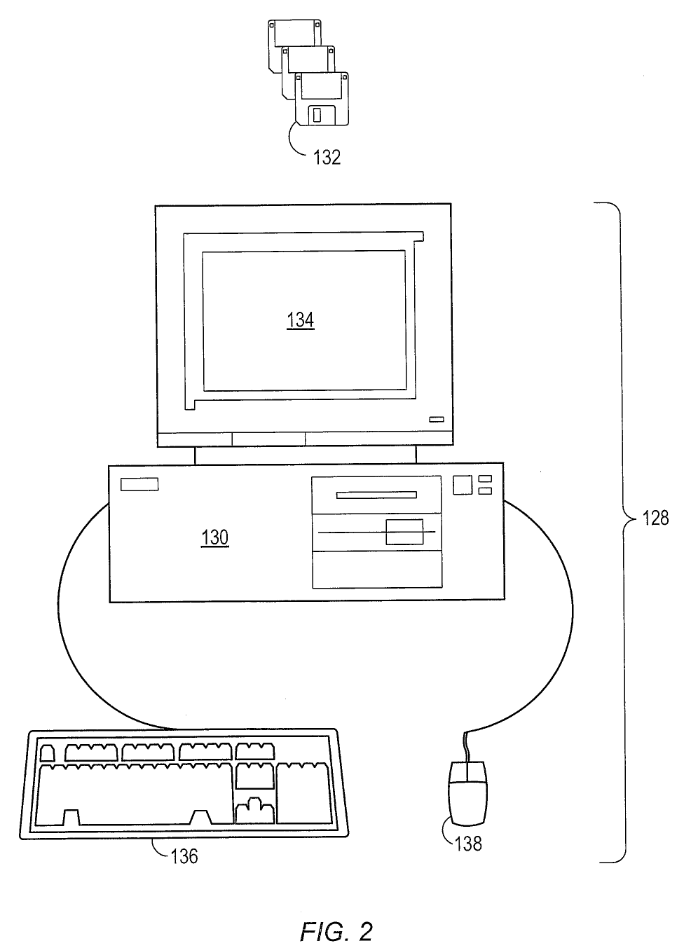

FIG. 2 illustrates an embodiment of computer system 128 that may be suitable for implementing various embodiments of a system and method for restricting the use of secure information. Each computer system 128 typically includes components such as CPU 130 with an associated memory medium such as floppy disks 132. The memory medium may store program instructions for computer programs. The program instructions may be executable by CPU 130. Computer system 128 may further include a display device such as monitor 134, an alphanumeric input device such as keyboard 136, and a directional input device such as mouse 138. Computer system 128 may be operable to execute the computer programs to implement a method for facilitating cardiac intervention as described herein.

Computer system 128 may include memory medium on which computer programs according to various embodiments may be stored. The term “memory medium” is intended to include an installation medium, e.g., a CD-ROM, or floppy disks 132, a computer system memory such as DRAM, SRAM, EDO RAM, Rambus RAM, etc., or a non-volatile memory such as a magnetic media (e.g., a hard drive or optical storage). The memory medium may also include other types of memory or combinations thereof. In addition, the memory medium may be located in a first computer that executes the programs or may be located in a second, different computer that connects to the first computer over a network. In the latter instance, the second computer may provide the program instructions to the first computer for execution. In addition, computer system 128 may take various forms such as a personal computer system, mainframe computer system, workstation, network appliance, Internet appliance, personal digital assistant (“TDA”), television system, or other device. In general, the term “computer system” generally refers to any device having a processor that executes instructions from a memory medium.

The memory medium may store a software program or programs operable to implement a method for restricting the use of secure information as described herein. The software program(s) may be implemented in various ways, including, but not limited to, procedure-based techniques, component-based techniques, and/or object-oriented techniques, among others. For example, the software program(s) may be implemented using ActiveX controls, C++ objects, JavaBeans, Microsoft Foundation Classes (“MFC”), browser-based applications (e.g., Java applets), traditional programs, or other technologies or methodologies, as desired. A CPU such as host CPU 130 executing code and data from the memory medium may include a means for creating and executing the software program or programs according to the methods and/or block diagrams described herein.

MIP is a simple three-dimensional visualization tool that can be used to display computed tomographic angiography data sets. MIP images are not threshold dependent and preserve attenuation information. Thus, they often yield acceptable results even in cases in which shaded surface display images fail because of threshold problems. MIP is particularly useful for depicting small vessels. Because MIP does not allow for differentiation between foreground and background, MIP images are best suited for displaying relatively simple anatomic situations in which superimposition of structures does not occur (e.g., the abdominal aorta). If anatomic structures are superimposed over the vessel of interest, the MIP technique can provide images of diagnostic quality as long as the contrast of the vessel of interest is sufficiently high compared with that of surrounding structures. Editing procedures for MIP are usually used to exclude unwanted structures from the region of interest and include cutting functions and region-growing algorithms. Artifacts from vessel pulsation and respiratory motion may occur and simulate abnormalities. MIP images should always be interpreted together with the original transaxial data set. Knowledge of display properties and artifacts is necessary for correct interpretation of MIP images and helps one create images of optimal quality, choose appropriate examination parameters, and distinguish artifacts from disease.

The MIP algorithm is commonly used as a three-dimensional post-processing method to depict volumetric vascular data sets acquired with both computed tomography (CT) and magnetic resonance imaging. Both modalities tend to produce a large number of primary reconstructed sections, which has prompted a greater use of three-dimensional post-processing. In addition, three-dimensional vascular anatomy is difficult to discern when only cross-sectional images are used. MIPs are capable of presenting angiogram-like views calculated from the primary data that make anatomic and pathologic features easier to identify.

MIP is a simple volume-rendering technique. For a given viewing direction, parallel rays are cast through a region of interest (ROI), and the maximum CT number encountered along each ray is displayed. This ROI may be determined from a stack of transaxial spiral CT images. For CT angiography, various editing procedures are used to exclude structures that might be superimposed over the vessel of interest. Bones usually have a higher CT number than contrast material-enhanced vessels and will be preferentially displayed on MIP images. Thus, exclusion of bones is necessary for most applications of CT angiography.

To produce MIPs, a viewing angle is chosen to define the projection plane. Parallel rays are then cast from the projection plane through the stack of reconstructed sections that make up the data volume, and the maximum intensity encountered along each ray is placed into the projection plane to construct the MIP. Vessels have higher contrast intensity values than those for soft tissue; therefore, the MIP shows a projected two-dimensional view of the vessels as seen from the center of the projection plane. Since some information is lost in the conversion from three to two dimensions, MIPs can be computed from many viewing angles and shown in a cine loop to convey the three-dimensional anatomy of the vessels.

The contrast in MIPs decreases with increasing projected volume (MIP thickness) because the probability that the maximum value encountered in the background will match or exceed the vessel intensity increases with MIP thickness. Although MIPs exhibit an increased contrast-to-noise ratio compared with that of source images, predominantly as a result of decreased noise, the reduced contrast between vessels and background can result in artifacts. This effect can lead to the disappearance of vascular features that have intensities only as great as the intensity of the background. Therefore, small vessels, which have decreased intensity as a result of volume averaging, can become invisible. The edges of larger vessels, which are less intense than the vessel center because of volume averaging, may be obscured, which leads to apparent vessel narrowing. High-grade stenoses may be overestimated on MIPs and appear as segmental vessel occlusions.

Regions of interest (ROIs) can be defined around vessels to limit the MIP thickness, thereby improving contrast in the MIP. In CT angiography, this method also allows the exclusion of high-attenuating bone that otherwise could overlap and obscure the vessels. A rectangular oblique plane can be easily specified and thickened to enclose a cubboidal ROI that can be used to produce conventional rectangular-slab MIPs, which are also known as thin-slab MIPs. In regions of complex and tortuous anatomy and for certain viewing angles, however, cuboidal ROIs cannot maximally exclude bone and may include excessive soft tissue. Usually, separate cuboidal ROIs have to be specified for each vessel of interest, which increases the number of MIP reconstructions per study. Alternatively, manual section-by-section editing can be performed to draw ROIs around structures to exclude or include them, but this is tedious, may not be reproducible, and may be susceptible to tracing errors.

Data editing can be avoided if only a few transaxial images are used to produce MIP images (a technique known as thin-slab MIP) in a caudocranial viewing direction. For interactive viewing, this slab can then be moved through the whole stack of transaxial images (i.e., sliding thin-slab MIP images).

In contrast to MIP, SSD requires the definition of a three-dimensional binary object. This object is then illuminated by a virtual light source, and the resulting reflections from the object surface determine the local gray values on the SSD image.

SSD images contain depth information about the object surface (foreground and background discrimination), but most SSD variants do not retain attenuation information from inside an object. In contrast, MIP images do not provide depth information, but they do contain attenuation information (eg, about vascular calcifications). Although SSD requires precise definition of the vessel of interest, MIP needs to exclude only disturbing overlying structures from the ROI to produce diagnostically useful images.

Differentiation between foreground and background is not possible on a single MIP image. On an MIP image, the voxel with the highest CT number is displayed, independent of the voxel position along the projecting ray. As a consequence, various projection effects occur. To achieve a three-dimensional effect, one must view multiple MIP images from slightly varying viewing angles (cine display).

Whenever the projecting ray hits a contrast-enhanced voxel, that voxel is displayed preferentially over voxels of soft-tissue attenuation values. Thus, concave regions may be superimposed by surrounding voxels, depending on the viewing direction. This “silhouette effect” produces a shadowlike image. Because of this projection effect, MIP images are well suited for display of simple vascular anatomy (eg, the abdominal aorta) but are not useful for visualization of complex anatomic situations with superprojecting vessels.

MIP images do not allow visualization of hypoattenuating intraluminal abnormalities. Intraluminal thrombi or pulmonary emboli can be detected only if they are directly adjacent to the vessel wall or if the CT numbers of the remaining contrasted vessel lumen are reduced because of partial volume averaging. In MIP images of an aortic dissection in which the true and false channels are enhanced to the same degree, dissecting membranes must tie parallel to the viewing direction to be directly visualized. Curved membranes cannot be seen. If there is a perfusion difference, however, MIP is sensitive in the depiction of the aortic dissection, but the width of the channel with the higher CT numbers (usually the true channel) will usually be overestimated.

The vascular contrast against the background attenuation determines the vessel size in the MIP image. For a given anatomic area, the background attenuation does not grow much with increasing vascular enhancement, as long as parenchymal organs and overlying vessels are excluded from the ROI. Under these conditions, the diameter of a vessel depends solely on the vascular contrast; that is, the difference in attenuation between vessel and background.

Vessel contrast depends on the parameters used for injecting the contrast material; vascular enhancement increases with flow rate and concentration of the contrast medium. However, vascular enhancement depends even more greatly on cardiac output and the resulting dilution effects: A high output (such as occurs in young or anxious patients) reduces vascular enhancement, whereas a low cardiac output (such as occurs in older patients or those with left-sided heart failure) increases the enhancement.

Vessel contrast on MIP images also depends on partial volume averaging effects. Partial volume averaging most strongly affects small vessels that run parallel to the scan plane. As a result, vessel contrast may be markedly reduced if the chosen effective section thickness considerably increases the vessel size.

In cases in which the vessel of interest will be subject to strong partial volume averaging (such as accessory renal arteries in MIP images of the abdominal aorta), one must attempt to achieve the highest possible vascular contrast. For MIP images of the neck, chest, pelvis, and extremities, vascular contrast is less critical.

Many of the problems associated with MIP algorithms may be overcome by gathering more data such that defects and inconsistencies may be averaged out. For example, as previously mentioned vessels are moving, pulsating human organs due, at least in part, to blood being conveyed through the vessel. Typically a three-dimensional image is recorded over a specific time frame, and MIP algorithms are used to transform the recorded three-dimensional image into a two-dimensional image. As technology has improved the amount of data gathered during a typical scan has increased while the time frame required gathering said data has decreased. While over all this has been very beneficial for patients (e.g., decreasing their discomfort due to at least, the decreased time required to gather data), this may have unintentionally increased the occurrence of certain artifacts and “false positives” (e.g., as relates to the assessment of blockages in blood vessels).

The shortened time frame from which data is recorded may lead to the normal movement of healthy vessels being assessed as blockages. Data gathered over extended time frames in some cases averaged out this movement leading to fewer false positives due to this particular reason.

In some embodiments, a method may include recording data in an ROI over four dimensions. Recording data over four dimensions may include recording data over the physical three-dimensions as well as recording the three-dimensional space over time. An ROI may include at least a portion of a cardiovascular system (e.g., human). An ROI may include, more specifically, at least a portion of a heart and/or the portion of the cardiovascular system associated with the heart.

In some embodiments, parallel rays are cast from a projection plane through a stack of reconstructed sections that make up a data volume from a CT scan (discussed herein), and the maximum intensity encountered along each ray is placed into the projection plane to construct a MIP. The reconstructed sections or slices are obtained from a three-dimensional CT image of at least a portion of a human body. Rays cast through the slices of the three-dimensional image gather the maximum intensity pixel or voxel and construct a MIP. The two-dimensional image may display a map of a portion of a system of lumens in a human body (e.g., blood vessels). The constructed MIP may be a two-dimensional image.

In some embodiments, a method may include facilitating a more accurate representation of an ROI (e.g., at least a portion of a system of blood vessels). Normal movement of an ROI (e.g., blood vessels) may be interpreted as abnormalities (e.g., unnatural constrictions or blockages) in the ROI. This is a common problem associated with MIPs. To overcome this problem a method may include gathering or providing additional three-dimensional images of a ROI. Additional images of a ROI may be obtained at different time periods. Images of a ROI from a different time period may be used to determine if an assessed abnormality is real physical abnormality in a subject or an artifact of the first image from which the MIP was generated.

A portion of a first image of an ROI containing assessed abnormalities may be compared to a corresponding portion of a second image of an ROI. Intensities from the portion of the first image and the portion of the second image may be assessed or compared to one another. Based upon the assessment of the intensities from the two portions one of the portions may be chosen to construct a new reassessed MIP image. This process may be repeated over and over as necessary in order to refine an MIP to overcome abnormalities associated with MIPs constructed from a limited data set (e.g., one three-dimensional image).

Any appropriate mathematical operator or algorithm may be used to assess the portions of two or more images in order to choose the portion more likely to be representative of a real physical state of a subject. In some embodiments, a greater than operator may be used to determine which of two or more corresponding portions of two or more images has the greater intensity relative to one another. The portion with the greater intensity may be chosen to form a portion of a reassessed MIP. In some embodiments, a mathematical operator may include a less than or equal to operator.

Although explanations of a method to this point include two three-dimensional images obtained at two different time periods, this should be viewed as exemplary only. In fact, the more images of an ROI obtained at different time periods the more accurate the resulting MIP will be. As CT scanning methods and systems improve so will the amount and quality of data improve. Naturally with this progression, the number of three-dimensional images which may be obtained within a given time frame will increase. The evolution of computed tomography from a device that required over 2 minutes to create a single poor-resolution image slice to one in which multiple slices can be obtained in less than 1 second and images displayed in a variety of presentations (multi-planar and 3-D) has propelled that technique into the forefront in the diagnosis of arterial vascular disease.

The method may include creating an image of at least a ROI using the resulting MIP. The created image may include a two-dimensional image in which heart blood vessels are highlighted (e.g., showing up as brighter areas relative to surrounding tissue). Traditionally an MIP converts a three-dimensional image into a two-dimensional image. Methods described herein may convert four-dimensional images to a three-dimensional image and/or a two dimensional image. In some embodiments, a four-dimensional image may include a plurality (e.g., a sequential series) of three-dimensional images of the same ROI acquired at different time periods.

The method as described to this point should not be seen as limiting. In some embodiments, a method may include creating an image of a ROI by assessing a property of corresponding portions of a plurality of multi-dimensional images of an ROI at different time periods. Intensity is but one example of a property of a portion of an image which may be assessed.

The order or manner in which these corresponding portions may be assessed in the described method should not be seen as limiting. Corresponding portions of multi-dimensional images (e.g., three-dimensional images) of a ROI obtained at different time periods may be assessed relative to one another automatically for the entire ROI. Assessment of a portion using corresponding portion from other images may not be reserved only for portions which include an assessed abnormality. Assessing all portions of an image of a ROI using corresponding portions from other images may make the assessment of abnormalities in an MIP unnecessary. Assessing portions of an image or a ROI may be performed in a method before during or after an MIP is being constructed.

Creating an MIP from a plurality of three-dimensional images of a ROI taken over a specified time period may result in a clearer more accurate MIP of a system of lumens in a human body. This more accurate MIP may allow for more accurate assessment of real physical abnormalities within a subject and specifically within the ROI of a subject. Additionally ROI may be a dynamic. A ROI may include a fixed number of voxels, which is static or it can be dynamic (e.g., size and/or shape of ROI may be changing). In some embodiments, certain voxels in the matrix or ROI may change with each phase based on additional information such as curvature, shape, pixel intensity and other factors which may modify ROI.

In some embodiments, a method may include assessing blockages in blood vessels in a human body. In specific embodiments the existence or lack thereof of blockages may be assessed in at least a portion of a human heart. Blockages may be caused by natural or unnatural means. Although many of the examples discussed herein may refer to blockages within a human heart, this should not be seen as limiting. A method may assess any type of blockage and/or restriction in any portion of a body lumen.

A method may include providing one or more digital images to a computer system. Digital images may be acquired using computed tomography imaging, magnetic resonance imaging, etc. The method used to acquire images may provide digital images. In some cases methods may be used to acquire images of a portion of a body which do not traditionally provide digital images (e.g., X-rays). In such cases a method may include digitizing an image or an image may be digitized in a separate operation before being provided to a computer system. There are many known methods for digitizing an image.

Images provided to a computer system may include multi-dimensional images. Images may be at least two-dimensional images. Images provided may include three or four-dimensional images. Images provided may include greater than four dimensional images. When referring to images and data associated with such images herein, dimensions should not be limited to only space and time. Dimensions may include other factors associated with a subject or a portion of a subject (e.g., the portion of the subject captured in the image provided). Dimensions may include factors including, but not limited to, area of contractile tissue; area of tissue potentially recoverable; area of tissue unlikely to be recoverable; percentage of contractile LAD; percentage of LAD potentially recoverable; percentage of LAD unlikely to be recoverable; and percentage of contractile LCX.

In some embodiments, an image may be adjusted to increase or reduce the amount of data included within the image as part of a method or prior to carrying out the method described herein. For example, dimensions may be added and/or subtracted to an image. In some embodiments, a series of two-dimensional images may converted to a three-dimensional image.

In some embodiments, at least one three-dimensional image may be provided to a computer system. One or more of the images may be of at least a portion of a human body (e.g., a human heart). The image may include pictures of one or more body lumens (e.g., blood vessel).

In some embodiments, a virtual cell may be positioned within a portion of a blood vessel depicted in at least one of the images. A cell may be formed from an arbitrary virtual volume. A volume of a cell may remain constant throughout the method as the method is carried out.

A cell may be positioned by a user within a blood vessel within the image. A cell may be positioned automatically by a computer system. A volume and/or a shape of a cell may be initially adjusted to fill a portion of a blood vessel such that the cell is contacting the walls of the blood vessel within the image. A cell may be composed of a number of voxels.

In some embodiments, a cell may be virtually moved through a blood vessel depicted in at least one of the provided images. A cell may be virtually moved by a user or by a computer system along the confines of the depicted blood vessel. Throughout the movement of the virtual cell through the depicted blood vessel, a volume of the cell may remain constant.

A virtual cell may be employed to detect blockages within a blood vessel depicted within an image. A computer system may assess positions along a blood vessel wherein a cell changes from a first shape to a second shape. Keeping a cell's volume constant as the cell is moved through a blood vessel may force the cell to change shape as it moves through a blood vessel in order to stay within the boundaries of the blood vessel. A cell may be forced to change shape when confronted with a blockage within the blood vessel restricting the blood vessel. The cell may change shape from a first shape to a second shape in order to move beyond the blockage while maintaining a constant volume. A computer system may assess positions within at least one of the images where the cell changes shape.

After moving through a position in a blood vessel where a blockage is located, a cell may change shape from a second shape to a third shape. The third shape may be at least roughly equivalent to the second shape, in that once the cell has moved past the blockage the blood vessel may have an equivalent cross-section to that of the blood vessel before the blockage, which the cell would then assume.

In some embodiments, two or more three-dimensional images of at least a portion of the human body may be provided to a computer system. The images may include at least a first image and a second image which are acquired at different time frames. Blockages assessed in a first image may be verified using at least a second image acquired at a different time of the portion of the body.

Due to the natural movements of the body, and especially of blood vessels, false positives of potential blockages of blood vessels can be common. Verifying assessed blockages may eliminate or at the very least reduce the occurrence of false positives during the assessment of blockages.

Verification may occur by assessing any changes in shape of a cell at an equivalent position in a blood vessel in the second image in a different phase. The position being equivalent to an assessed position of the blockage in the first image. If a blockage is assessed in the second image at the same position but during a different time frame, then the blockage has been verified. However, if the blockage is not verified in at least a second image then the blockage may not be indicated to a user.

In some embodiments, an assessed blockage may be verified using two or more additional images. One or more of the additional images may have been acquired during a different time frame than the first image.

In some embodiments, a method may include creating an image. A created image may depict assessed positions along the blood vessels where a blockage has been detected. Blockages may be depicted in any of a number of known methods including, but not limited to, highlighting and/or outlining in color or grayscale. Severity of a blockage may be assessed and depicted in created images accordingly. The created image may allow a user to see where assessed blockages are positioned within a human heart. Created images may be two-dimensional. Created images may at least appear three or four-dimensional.

There are many methods for determining models and borders of features (e.g., blood vessels) from digital images. Of the different methods available to assist in creating finite element models, several of the methods may be divided into several categories. For example, methods may differ in what aspect of provided data (e.g., images) the methods operate on. For example, in some embodiments, a method may operate on the density and/or intensity of an image. In certain embodiments, a method for creating finite element models may operate on the boundaries and/or the gradient of an image.

In some embodiments, methods of creating finite element models may differ in their initialization. For example, some methods may require an initial solution. An initial solution may take the form of user input. User input may include, for example, a user assessing and/or identifying a particular heart feature and/or portion of a heart feature within an image of human heart tissue. In some embodiments, a method of creating finite element models may not require an initial solution and therefore may be considered self-initialized (i.e., fully automated). In some embodiments, a self-initialized method may provide a required initial solution for a method that is not fully automated (e.g., a method which typically requires user input).

In certain embodiments, methods for creating finite element models and/or segmenting data (e.g., an image) may include methods that operate on the boundaries and/or gradient of an image (i.e., boundary based methods). Boundary based methods estimate the boundaries in an image. Boundary based methods may estimate the boundaries in an image based on the contrast between adjacent pixels of a digitized image. Based on this contrast between adjacent pixels, a border of a structural feature to be assessed may be extracted from among all of the borders detected by the boundary based method. General descriptions of some examples of known boundary based methods are described herein; however, the examples and their descriptions should not be viewed as limiting. Many of the same output products may be arrived at by a variety of mathematical operators known to one skilled in the art and those provided here are merely illustrative examples.

In some embodiments, detection of the border may be accomplished using gradients. A gradient for each point in an image may provide several pieces of information. For example, the gradient may provide the “strength” of the border. The strength of the border may be represented by a magnitude of a vector. The gradient may provide a direction to a maximum brightness. The direction to the maximum brightness may be represented by the direction of the vector.

In some embodiments, gradients may be assessed using the Sobel operation, otherwise known as the Sobel Edge Detector. In brief, the Sobel operation performs a 2-D spatial gradient measurement on an image and thus emphasizes regions of high spatial gradient that correspond to edges. Typically the Sobel operation is used to find the approximate absolute gradient magnitude at each point in an input grayscale image. Methods of determining models and borders of features from digital images are described in U.S. patent application Ser. No. 11/342,296 entitled “METHOD AND SYSTEM FOR IMAGE PROCESSING AND ASSESSMENT OF A STATE OF A HEART” and filed on Jan. 27, 2006, and is herein incorporated by reference.

More and more advances in imaging technology have led to the ability of users to gather more data and at a much faster rate on one or more portions of a subject. This increasing ability has been a boon to the medical industry as well as greatly increasing the quality of care for subjects. With the ability to gather data at a faster rate problems have arisen. The ability to acquire highly detailed multi-dimensional digital images of subjects has lead to problems with storing and/or transferring this data easily. The ability to communicate large amounts of data between remote sites has not kept up with medical imaging's ability to acquire large amounts of data.

Users must have the ability to transfer data to other users easily that may have limited access to large bandwidth Internet access. A method of facilitating transfer of data between remote sites is needed.

In some embodiments, a method may be provided which facilitates transfer of data between sites. A method may facilitate transfer of data related to blockages in human lumens. Lumens may include blood vessels. Specific embodiments may include blood vessels positioned within a human heart.

In some embodiments, a method of facilitating transfer of data may include providing one or more images of at least a portion of a human body. A method may include providing one or more digital images to a computer system. Digital images may be acquired using computed tomography imaging, magnetic resonance imaging, etc. The method used to acquire images may provide digital images. In some cases methods may be used to acquire images of a portion of a body which do not traditionally provide digital images (e.g., X-rays). In such cases a method may include digitizing an image or an image may be digitized in a separate operation before being provided to a computer system. There are many known methods for digitizing an image.

Images provided to a computer system may include multi-dimensional images. Images may be at least two-dimensional images. Images provided may include three or four-dimensional images. Images provided may include greater than four dimensional images. When referring to images and data associated with such images herein, dimensions should not be limited to only space and time. Dimensions may include other factors associated with a subject or a portion of a subject (e.g., the portion of the subject captured in the image provided). Dimensions may include factors including, but not limited to, area of contractile tissue; area of tissue potentially recoverable; area of tissue unlikely to be recoverable; percentage of contractile LAD; percentage of LAD potentially recoverable; percentage of LAD unlikely to be recoverable; and percentage of contractile LCX.

In some embodiments, an image may be adjusted to increase or reduce the amount of data included within the image as part of a method or prior to carrying out the method described herein. For example, dimensions may be added and/or subtracted to an image. In some embodiments, a series of two-dimensional images may be converted to a three-dimensional image.

In some embodiments, at least one three-dimensional image may be provided to a computer system. One or more of the images may be of at least a portion of a human body (e.g., a human heart). The image may include pictures of one or more body lumens (e.g., blood vessel).

In some embodiments, a method may include assessing blockages in lumens in a human body using one or more of the images. Methods for assessing blockages in lumens (e.g., blood vessels) are described herein.

In some embodiments, a method may include creating an image. A created image may depict assessed positions along the blood vessels where a blockage has been detected. Blockages may be depicted in any of a number of known methods including, but not limited to, highlighting and/or outlining in color or grayscale. Severity of a blockage may be assessed and depicted in created images accordingly. The created image may allow a user to see where assessed blockages are positioned within a human heart. Created images may be multi-dimensional. Created images may be two-dimensional. Created images may at least appear three or four-dimensional.

In some embodiments, a method may include reducing the resolution of portions of the created image. Reducing the resolution of one or more portions of a created image may reduce the amount of data associated with the image. Reducing the resolution of portions of a created image may not reduce the value of the created image to a user or client. Portions of the created image of which the resolution is reduced may be selected so as not to reduce the value of the image.

For example, using a method described herein, blockages may be assessed from a four-dimensional image (the fourth dimension being time) of a human heart. The method may create a four-dimensional image of the human heart depicting the assessed blockages. In the current example the assessed blockages may be determined to be the information most valued contained within the created image. The depicted assessed blockages may then be kept at a high resolution while the resolution of the rest of the created image may be decreased, effectively decreasing the bandwidth required to transfer the created image between remote sites.

In some embodiments, a method of facilitating transfer of data may include reducing a number of dimensions included in a created image. For example, looking at the previously described example, wherein a four-dimensional image was created, at least one of the dimensions may be removed to decrease the amount of data associated with the created image. In the example described in the previous paragraph, the fourth dimension of time may be removed to reduce the data load. A user, for example, may determine that the dimension of time is unnecessary in order to depict the assessed blockages, such that a two or three-dimensional image is enough to convey the necessary data.

In some embodiments, one or more portions of the method may be performed by a computer system. As such one or more portions of the method may be automated or semi-automated. For example a user may decide which portions of a created image are important and which are unimportant. In some embodiments, a computer system may decide which portions of a created image to reduce the resolution for. Similarly the user or system may select one or more portions of an image from different phases. The portions may be combined to create a new image. From this new image ROIs may be identified.

Cardiac calcium scoring uses a computed tomography scan to find the buildup of calcium on the walls of the arteries of the heart (coronary arteries). This test may be used to check for heart disease in an early stage and to determine how severe it is. Cardiac calcium scoring is also called coronary artery calcium scoring. The coronary arteries supply blood to the heart. Normally, the coronary arteries do not contain calcium. Calcium in the coronary arteries is a sign of Coronary Artery Disease (CAD). A CT scan takes pictures of the heart in thin sections. The pictures are recorded in a computer and can be saved for more study or printed out as photographs.

Cardiac calcium scoring may be performed to check for early heart disease or to find out how severe heart disease is.

A cardiac calcium scoring test is usually done by a radiology technologist. The pictures are usually interpreted by a radiologist or a cardiologist. Other doctors, such as a family medicine doctor, internist, cardiologist, or surgeon, may also review a cardiac calcium scoring test.

Typically electrodes will be positioned on a subject's chest. Wires connect these to an EKG machine that records the electrical activity of the subject's heart on paper. The EKG records when the subject's heart is in the resting stage, which is the best time for the CT scans to be taken.

If the subject's heart rate is 90 beats per minute or higher, the subject may be given a drug to slow the subject's heart rate. The preferred heart rate is to be below 60 beats per minute.

During the test, the subject may lie on a table connected to the CT scanner. The scanner is a large doughnut-shaped machine.

The table slides into the round opening of the machine and the scanner moves around the subject's body. The table will move a little every few seconds to take new pictures.

The subject may be asked to hold the subject's breath for 20 to 30 seconds while about 200 pictures of the subject's heart are taken. It is very important to hold completely still while the pictures are taken.

During the test, the subject is usually alone in the scanner room. However, the technologist will watch the subject through a window. The subject may be able to talk to him or her through a speaker.

There is always a slight risk from being exposed to any radiation, including the low levels used for a CT scan.

Cardiac calcium scoring uses a CT scan to find the buildup of calcium on the walls of the arteries of the heart (coronary arteries). The a user may discuss initial results of the cardiac calcium scoring test with the subject right after the test.

Cardiac calcium scoring

| Score | Presence of plaque |
|---|---|
| 0 | No plaque is present. The subject has less than a 5% chance of |
| | having heart disease. The subject's risk of a heart attack is |
| | very low. |
| 1-10 | A small amount of plaque is present. The subject has less than a |
| | 10% chance of having heart disease. The subject's risk of a heart |
| | attack is low. However, the subject may want to take precautions |
| | (e.g., quit smoking, eat better, and exercise more). |
| 11- | Plaque is present. The subject's has mild heart disease. The |
| 100 | subject's chance of having a heart attack is moderate. The |
| | subject maybe should consider talking with a doctor about |
| | quitting smoking, eating better, beginning an exercise program, |
| | and any other treatment the subject may need. |
| 101- | A moderate amount of plaque is present. The subject jasheart |
| 400 | disease, and plaque may be blocking an artery. The subject's |
| | chance of having a heart attack is moderate to high. The |
| | subject's health professional may want more tests and may start |
| | treatment. |
| Over | A large amount of plaque is present. The subject has more than a |
| 400 | 90% chance that plaque is blocking one of the subject's arteries. |
| | The subject's chance of having a heart attack is high. |

The higher the subject's score on cardiac calcium testing, the more plaque the subject has in the arteries of the subject's heart. This makes the subject's chance of having a heart attack higher.

Plaque that is not hard (soft plaque) cannot be found with cardiac calcium scoring. Soft plaque is fat buildup within the walls of the arteries of the heart. If a subject has soft plaque in the subject's arteries, the test may look normal because the lumen is open but within the wall there is soft plaque buildup but this is a false-negative result. Soft plaque may also cause a heart attack.

Currently calcium scoring may be recommended for men age 45 and older and women age 55 and older who have a higher chance of heart disease. Younger adults may be tested if they have a very strong family history of heart disease.

If the subject's cardiac calcium scoring shows that the subject has a high chance of having heart disease, the subject may take steps to lower the subject chance (e.g., eat better, quit smoking, and get more exercise).

It is possible to have a false-positive test. This means that the test may show a high chance of blockage in the arteries of the heart when it is not true. People with a low chance of heart disease are most likely to have a false-positive test.

Calcium scoring is a useful test as regards assessing a subject's risk of a heart attack and generally checking the over all health of a subject's heart. However, currently interpretation of data by medical staff is difficult and time consuming, limiting the usefulness of the technique. Difficulties arise due to the abundance of calcium throughout the human body, not just in blood vessels. For example, large quantities of calcium deposits may be seen in the mitrial valve annulus and leaflets, in the left ventricular cavity, or in the aortic valve, sometimes making it difficult to locate and assess the minor calcium deposits in blood vessels in and/or around the heart. Location of calcium deposits in the heart in a digital image using calcium scoring is typically done manually. There is a need for a semi-automated or automated method for finding a position of a coronary within a calcium scoring image.

In some embodiments, a method of imaging calcium in blood vessels in a human heart may include at least a first image of at least a portion of a human body to a computer system. A method may include providing one or more digital images to a computer system. Digital images may be acquired using computed tomography imaging, magnetic resonance imaging, etc. The method used to acquire images may provide digital images. In some cases methods may be used to acquire images of a portion of a body which do not traditionally provide digital images (e.g., X-rays). In such cases a method may include digitizing an image or an image may be digitized in a separate operation before being provided to a computer system. There are many known methods for digitizing an image.

Images provided to a computer system may include multi-dimensional images. Images may be at least two-dimensional images. Images provided may include three or four-dimensional images. Images provided may include greater than four dimensional images. When referring to images and data associated with such images herein, dimensions should not be limited to only space and time. Dimensions may include other factors associated with a subject or a portion of a subject (e.g., the portion of the subject captured in the image provided). Dimensions may include factors including, but not limited to, area of contractile tissue; area of tissue potentially recoverable; area of tissue unlikely to be recoverable; percentage of contractile LAD; percentage of LAD potentially recoverable; percentage of LAD unlikely to be recoverable; and percentage of contractile LCX.

In some embodiments, an image may be adjusted to increase or reduce the amount of data included within the image as part of a method or prior to carrying out the method described herein. For example, dimensions may be added and/or subtracted to an image. In some embodiments, a series of two-dimensional images may be converted to a three-dimensional image.

In some embodiments, at least one three-dimensional image may be provided to a computer system. One or more of the images may be of at least a portion of a human body (e.g., a human heart). The image may include pictures of one or more body lumens (e.g., blood vessel).

In some embodiments, a method may include assessing a position of blood vessels of a human heart within a first image. The first image may include a three-dimensional C positive image. A position of blood vessels may be assessed by assessing an intensity in a plurality of voxels from the first image. A method may include providing at least a second image of at least a portion of the human body. The first image may include a three-dimensional C negative image or Calcium scoring study image. A method may include assessing a position of the heart within the second image using the assessed position of blood vessels in the first image. Additionally, a smart scaling and shifting, or shape based algorithms may be used to correct for any motion that may have occurred between the two acquisitions.

In some embodiments, a method may include assessing calcium within the blood vessels associated with the heart. In some embodiments, a method may include creating an image. A created image may depict calcium within the blood vessels of the heart from the second image. Calcium may be depicted in any of a number of known methods including, but not limited to, highlighting and/or outlining in color or grayscale. Severity of a calcium buildup and/or type of calcium may be assessed and depicted in created images accordingly. The created image may allow a user to see where assessed calcium are positioned within a human heart. Created images may be multi-dimensional. Created images may be two-dimensional. Created images may at least appear three or four-dimensional.

Broadly speaking there are currently two forms of cardiac imaging. One example of cardiac imaging may be referred to as coronary images. Coronary images typically include images generated by various means shortly after a contrast agent has been administered to a subject, introducing the contrast agent into the subject's blood stream. Images acquired in this manner display blood vessels and the chambers of the heart as well as any place in which blood flows through a body. A second example of cardiac imaging may be referred to as viability images. Viability images typically include images generated by various means after a contrast agent has been administered to a subject, introducing the contrast agent into the subject's blood stream. To obtain viability images, typically there is a necessary delay between introduction of a contrast agent and the acquiring any images. The delay is to typically allow the contrast agent to permiate normal muscle tissue and wash out of it, while in infarcted tissue it takes much longer to wash out. Hence if imaged at appropriate time interval an image is captured where the infarcted tissue is high lighted

Currently coronary and cardiac images are acquired separately and typically reviewed and assessed separately. There is a need to combine these two types of cardiac images to provide a user with a more complete and accurate picture of a subject's heart to better assess a state of the heart. Combining the information from the two types of cardiac images would allow users to more accurately and efficiently assess a state of the heart.

In some embodiments, a method may combine coronary images and viability images. A method may include providing at least one coronary image of at least a portion of a human body to a computer system. At least one of the coronary images may include at least a portion of a heart. A method may include providing at least one viability image of at least a portion of a human body to a computer system. At least one of the viability images may include at least a portion of a heart. A method may include providing one or more digital images to a computer system. Digital images may be acquired using computed tomography imaging, magnetic resonance imaging, etc. The method used to acquire images may provide digital images. In some cases methods may be used to acquire images of a portion of a body which do not traditionally provide digital images (e.g., X-rays). In such cases a method may include digitizing an image or an image may be digitized in a separate operation before being provided to a computer system. There are many known methods for digitizing an image.

Images provided to a computer system may include multi-dimensional images. Images may be at least two-dimensional images. Images provided may include three or four-dimensional images. Images provided may include greater than four dimensional images. When referring to images and data associated with such images herein, dimensions should not be limited to only space and time. Dimensions may include other factors associated with a subject or a portion of a subject (e.g., the portion of the subject captured in the image provided). Dimensions may include factors including, but not limited to, area of contractile tissue; area of tissue potentially recoverable; area of tissue unlikely to be recoverable; percentage of contractile LAD; percentage of LAD potentially recoverable; percentage of LAD unlikely to be recoverable; and percentage of contractile LCX.

In some embodiments, at least one three-dimensional image may be provided to a computer system. One or more of the images may be of at least a portion of a human body (e.g., a human heart). The image may include pictures of one or more body lumens (e.g., blood vessel).

In some embodiments, a method may include combining at least one of the coronary images with at least one of the viability images. A method may include using at least one feature to spatially align at least one of the coronary images with at least one of the viability images.

In some embodiments, a feature may include an anatomical landmark. An anatomical landmark may include any portion of a human body visible using any medical imaging device. An anatomical landmark may include at least a portion of a spine. An anatomical landmark may include at least a portion of a rib. Features may include any easily identifiable portion of a human body depicted in both the coronary and viability images. For example, a rib or aorta or sternum depicted in both a coronary and viability image may function as a feature allowing a computer system to virtually align and overlay the virtual images.

A method may be automated or semi-automated. In some embodiments, a user may manually select a feature in at least two of the images to use as a feature. In some embodiments, a computer system may automatically select a feature in at least two of the images to use as a feature, to align both images.

In some embodiments, a method may include creating an image. A created image may depict at least some of the features depicted in at least one of the coronary images and at least one of the viability images. Features may be depicted in any of a number of known methods including, but not limited to, highlighting and/or outlining in color or grayscale. Severity of a problem or potential problem may be assessed and depicted in created images accordingly. The created image may allow a user to better assess a state of a human heart. Created images may be multi-dimensional. Created images may be two-dimensional. Created images may at least appear three or four-dimensional.

Sudden cardiac death in patients with coronary artery disease is predominantly caused by ventricular tachycardia (VT)/ventricular fibrillation (VF). Patients with a low left ventricular ejection fraction (LVEF) and inducible ventricular tachycardia during electrophysiologic study (EPS) are at risk of sudden death and may benefit from implantable cardioverterdefibrillator (ICD) therapy. Low left ventricular ejection fraction and ventricular tachycardia inducibility identify a substrate for ventricular tachycardia. Ventricular tachycardia occurs more commonly in the setting of larger infarcts, and left ventricular ejection fraction is inversely related to infarct size. EPS directly establishes the presence of a substrate by the actual induction of ventricular tachycardia. To date, there is only indirect information relating infarct size or morphology to the presence of a substrate for ventricular tachycardia in humans. Contrast-enhanced magnetic resonance imaging (ceMRI) with a gadolinium-based contrast agent has been shown to identify, with high precision, areas of myocardial infarction in both animals and humans. It has been hypothesized that infarct size and/or morphology detected by ceMRI is a better predictor of EPS inducibility of ventricular tachycardia than left ventricular ejection fraction.

Studies have demonstrated that infarct surface area and size, as measured by MRI, is a better identifier of patients who have a substrate for inducible MVT than left ventricular ejection fraction. In humans, limited information suggests that infarct size, as measured by left ventricular ejection fraction, maximum creatine kinase, and the number of fixed thallium defects, is related to induction of ventricular arrhythmias. It has been reported that patients with clinical ventricular tachycardia after myocardial infarction had larger infarcts than those without. Recently, extensive scar tissue detected by technetium-99m tetrofosmin scintigraphy was reported as an independent predictor of death or recurrent ventricular arrhythmias in survivors of aborted sudden death. Because improvements in ceMRI have allowed delineation of infarct regions with high precision, it was demonstrated that infarct size, measured in vivo, is an important predictor of induction of MVT during EPS.

The left ventricular ejection fraction is inversely related to infarct size, although the strength of this relationship may be poor. Many factors affect left ventricular ejection fraction aside from infarct size, such as preload, afterload, autonomic factors, medications, and post-infarction remodeling. Many of these may also influence the pathogenesis of ventricular tachyarrhythmias by affecting the substrate or by serving as triggers or modulating factors. As inducibility of ventricular tachycardia during EPS evaluates for the presence of a fixed substrate for ventricular tachycardia, it is not surprising that the factor most closely linked to the anatomic substrate—infarct size (surface area)—is a better discriminator of inducible ventricular tachycardia than left ventricular ejection fraction, which is affected by so many other variables. A recent study found that extensive scar tissue had a higher hazard ratio for recurrent events than left ventricular ejection fraction (2.4 vs. 2.0), although the definition of extensive scar tissue was not clearly stated.

The clinical significance of inducible PVT/VF has been the subject of controversy. Induction of PVT/VF may be a nonspecific response to aggressive stimulation, as it may be observed frequently in patients with normal hearts. Yet, the clinical significance of these arrhythmias might differ depending on the presence and severity of heart disease. These arrhythmias are inducible in a substantial percentage of patients who have survived cardiac arrest. Furthermore, in some patients, after treatment with anti-arrhythmic agents, MVT may be induced; it is therefore plausible that these patients have a fixed substrate for ventricular arrhythmias that, in the absence of anti-arrhythmic drugs, is polymorphic.

Studies demonstrate that characterization of infarct size is a better predictor than left ventricular ejection fraction for inducibility of ventricular tachycardia. Although inducibility of ventricular tachycardia is not the ideal risk stratifier for prediction of sudden death, left ventricular ejection fraction is a known strong predictor. If the role of left ventricular ejection fraction as a predictor of sudden death is a surrogate feature for infarct size, then it is possible that measurement of infarct size by ceMRI may be a better predictor of sudden death than left ventricular ejection fraction. Studies demonstrating that infarct surface area and size is a reliable identifier of patients who have a substrate for inducible MVT is described in Bello, D. et al., and is herein incorporated by reference.

As methods of data acquisition within the medical field has progressed, methods of assessment of this relative flood of data have lagged behind. A method of quantifying a metric of an indicator or a feature of a human heart is needed.

In some embodiments, a method may include assessing a state of a heart. A method may include providing one or more viability images of at least a portion of a human heart to a computer system. At least one of the viability images may include at least a portion of a heart. A method may include providing one or more digital images to a computer system. Digital images may be acquired using computed tomography imaging, magnetic resonance imaging, etc. The method used to acquire images may provide digital images. In some cases methods may be used to acquire images of a portion of a body which do not traditionally provide digital images (e.g., X-rays). In such cases a method may include digitizing an image or an image may be digitized in a separate operation before being provided to a computer system. There are many mown methods for digitizing an image.

Images provided to a computer system may include multi-dimensional images. Images may be at least two-dimensional images. Images provided may include three or four-dimensional images. Images provided may include greater than four-dimensional images. When referring to images and data associated with such images herein, dimensions should not be limited to only space and time. Dimensions may include other factors associated with a subject or a portion of a subject (e.g., the portion of the subject captured in the image provided). Dimensions may include factors including, but not limited to, area of contractile tissue; area of tissue potentially recoverable; area of tissue unlikely to be recoverable; percentage of contractile LAD; percentage of LAD potentially recoverable; percentage of LAD unlikely to be recoverable; and percentage of contractile LCX.

In some embodiments, at least one three-dimensional image may be provided to a computer system. One or more of the images may be of at least a portion of a human body (e.g., a human heart). The image may include pictures of one or more body lumens (e.g., blood vessel).

In some embodiments, a method may include calculating a quantitative metric using one or more features derived from one or more viability images of the human heart. Non-viable sectors may be automatically or semi-automatically identified based on pixel intensity or Hounsfield unit and various geometries may be assessed (e.g., area, mass, volume). Features may include a size of an infarct in a human heart. A size of an infarct may be at least partially defined as an area of an infarct. A size of an infarct may be at least partially defined as a mass of an infarct. A size of an infarct may be at least partially defined as a percentage of a ventricle size.

A feature may include an area of the infarct that is in contact with viable muscle. In some embodiments, at least one of the features may include a morphology of an infarct. A feature may include a ratio of viable but akinetic muscle to non-viable muscle.

In some embodiments, a method may include assessing a heart's risk factor of Sudden Cardiac Death. A heart's risk factor of Sudden Cardiac Death may be assessed using a calculated quantitative metric.

In some embodiments, a method may include assessing a heart's risk factor of V-tach. A heart's risk factor of V-tach may be assessed using a calculated quantitative metric.

Magnetic resonance imaging has become a powerful noninvasive tool to define occlusive and dilating conditions that affect the vasculature. Stronger, faster magnetic gradients, creative radiofrequency pulsing maneuvers, and faster computing techniques have contributed to this success.

Computed tomographic angiography (CTA) applies current helical technology with a sustained high flow of iodinated contrast material via intravenous injection. The resultant data can be processed into thin axial images (source images), as well as into three-dimensional or multiplanar images (or both). Before helical (spiral) scanners became available, CT provided minimal coverage and three-dimensional volume techniques were primitive. As in three-dimensional ceMRA, a relatively large volume can be covered in a single breath, which provides spatial resolution free of respiratory motion artifact.

Good CTA requires contrast agent to be present in the vascular system of interest throughout the time that the CT images are acquired. This is accomplished by beginning CT imaging when adequate contrast levels are present and by ensuring sustained contrast throughout the scan. Two techniques are used to determine the time at which scanning should begin, relative to the initiation of contrast injection. In the test bolus technique, multiple scans are obtained at a single area of interest after a small injection of contrast agent and the arrival time is calculated. In the 2nd technique, an automated bolus-tracking system begins scanning when the density or intensity of an area defined by the operator exceeds a prescribed threshold. Like MRA, CTA display comprises the actual scan slices, reconstructed thinner slices, and three-dimensional techniques: maximum intensity projection; shaded surface display, or SSD; and volume rendering, or VR. Reconstructed thinner slices (smaller than beam collimation) and three-dimensional techniques are generally produced on a workstation.

Recently, several manufacturers of CT equipment have introduced a new generation of CT scanners (multi-detector array or multislice) that enable 2 to 4 image slices to be obtained during a single revolution of the scanner (0.5 to 1.0 sec). This advance produces much faster CT studies, with resolution similar or superior to the resolution achieved by the older equipment. Moreover, areas 3 to 6 times larger can be scanned without significant image degradation. This advance will enable wider application of CT in the diagnosis of peripheral vascular disease.

Contrast-enhanced (CE) MRI can characterize acute myocardial infarction (MI) with two well-defined CE patterns as follows: (1) First-pass images performed immediately after contrast injection often demonstrate areas of reduced CE MRI or hypoenhancement in the endocardial core of the infarct, corresponding to microvascular obstruction; (2) Delayed images (e.g., 10 to 20 minutes after contrast injection) demonstrate regional signal hyperenhancement, corresponding to myocardial necrosis. It has been hypothesized that a combination of CE perfusion MRI with functional data might be useful for the identification of myocardial viability, allowing one to distinguish permanently dysfunctional myocardium from dysfunctional segments bound to recover contractile function and contribute to left ventricular (LV) stroke volume after MI. However, previous studies have provided conflicting data regarding the interpretation of these perfusion patterns for the identification of viable and nonviable myocardium in patients after MI.

Herein methods have been described for combining data from coronary and viability images after the images have been acquired in order to create a new imaging. The created image may include at least some of the data from both the coronary and viability images. However, acquiring these images requires exposing a subject to at least two large doses of radiation from an imaging device. A safer alternative would be to develop a method which required that a patient only be exposed to no more than one dose of radiation an acquire both coronary and viability images at the same time.

In some embodiments, a method may acquire computed tomography images of a human body. A method may include administering a first dose of contrast agent to a human body. In some embodiments, a method may include waiting a predetermined period of time. A method may include administering a second dose of contrast agent to the human body. A method may include acquiring at least one computed tomography image of at least a portion of the human body.

Contrast agents, sometimes referred to as “dyes,” are used to highlight specific areas so that the organs, blood vessels, and/or tissues are more visible. By increasing the visibility of all surfaces of the organ or tissue being studied, they can help a radiologist determine the presence and extent of disease or injury.

Contrast agents are available in several different forms, but in general a CT contrast agent is a pharmaceutical substance. Some of the more common contrast agents used may include, but are not limited to, Iodine, Barium, Barium sulfate and Gastrografin

In some embodiments, a first dose and/or a second dose of contrast agent may be administered orally, subcutaneously, percutaneously, and/or intravenously.

Contrast agents may be administered in four different ways: Intravenous injection, Oral administration, Rectal administration, and/or Inhalation. Inhalation is a relatively uncommon procedure in which xenon gas is inhaled for a highly specialized form of lung or brain imaging. The technique is only available at a small number of locations worldwide and is used only for rare cases.

Intravenous contrast is used to highlight blood vessels and to enhance the structure of organs like the brain, spine, liver, and kidney. The contrast agent (usually an iodine compound) is clear, with a water-like consistency. Typically the contrast is contained in a special injector, which injects the contrast through a small needle taped in place (usually on the back of the hand) during a specific period in the CT exam.

Once the contrast is injected into the bloodstream, it circulates throughout the body. The CT's x-ray beam is weakened as it passes through the blood vessels and organs that have “taken up” the contrast. These structures are enhanced by this process and show up as white areas on the CT images. When the test is finished, the kidneys and liver quickly eliminate the contrast from the body.

Oral contrast is used to highlight gastrointestinal (GI) organs in the abdomen and pelvis. If oral contrast will be used during an examination, the patient will be asked to fast for several hours before administration.

Two types of oral contrast used include, but are not limited to, barium sulfate and gastrografin. Barium sulfate, the most common oral contrast agent, resembles a milk shake in appearance and consistency. The compound, available in various flavors, is prepared by mixing with water. Gastrografin is a yellowish, water-based drink mixed with iodine. It can have a bitter taste.

When oral contrast has been requested by the doctor, patients usually drink about 1,000 cc to 1,500 cc (the equivalent of three or four 12-ounce drinks). After the contrast is swallowed, it travels to the stomach and gastrointestinal tract. Like intravenous iodine, barium and gastrografin weaken x-rays. On CT images, the organs that have “taken up” the contrast appear as highlighted white areas.

Rectal contrast is used when enhanced images of the large intestine and other lower GI organs are required. The same types of contrast used for oral contrast are used for rectal contrast, but in different concentrations.

Rectal CT contrast is usually administered by enema. When the contrast is administered, the patient may experience mild discomfort, coolness, and a sense of fullness. After the CT is complete, the contrast is drained and the patient may go to the bathroom.

The preparation for rectal contrast is similar to oral contrast, in that the patient should be fasting for several hours before the test. In addition, the patient will be required to use a Fleets Enema to cleanse the colon; it is usually used the night before the examination.

For the most part contrast agents are relatively safe such that administration of two doses is preferred to exposing a subject to the radiation required to perform two CT scans. In administering two doses of contrast agent at prescribed intervals one may be able to acquire both coronary and viability images at in a single CT scan.

In some embodiments, a predetermined period of time may range from 5 to 10 minutes, 2 to 15 minutes, 10 to 30 minutes, and/or 2 to 60 minutes. The delay allows the first dose of contrast agent to absorb into body tissue (e.g., cardiac muscle tissue) allowing acquirement of viability images. The second dose is administered shortly before scanning and is therefore still in the blood stream of the subject, allowing for the acquirement of coronary images.

In this patent, certain U.S. patents, U.S. patent applications, and/or other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1. A method of imaging blood vessels in a human body, comprising:
providing a plurality of three-dimensional images of at least a portion of a human body acquired over a period of time to a computer system, wherein the plurality of images comprises at least a first image and a second image acquired at different times;
dividing the first image and the second image into a plurality of regions, wherein each of the regions corresponds between the first image and the second image;
assessing a property in a plurality of regions of the body from the first image;
assessing the property in a corresponding region of the body from the second image; and
comparing the property of the regions of the body from the first image to the property of the regions of the body from the second image to select either a region from the first image or a corresponding region from the second image; and
creating a third image of at least a portion of human blood vessels using the selected regions.

2. The method of claim 1, wherein the first image and the second image comprise at least a portion of a human heart.

3. The method of claim 1, wherein a region comprises one or more voxels.

4. The method of claim 1, wherein a property comprises an intensity of a region.

5. The method of claim 1, wherein comparing the property of the regions comprises using a mathematical operator to compare the regions.

6. The method of claim 1, wherein comparing the property of the regions comprises using a mathematical operator to compare the regions, and wherein the mathematical operator comprises the operator greater than.

7. The method of claim 1, wherein the third image at least appears three-dimensional.

8. The method of claim 1, wherein the third image is two-dimensional.

9. The method of claim 1, wherein at least a portion of the plurality of three-dimensional images are acquired using computed tomography imaging.

10. The method of claim 1, wherein at least a portion of the plurality of three-dimensional images are acquired using magnetic resonance imaging.

11. A system, comprising:
a CPU; and
a system memory coupled to the CPU, wherein the system memory stores one or more computer programs executable by the CPU;
wherein one or more computer programs are executable to perform a method, comprising:
providing a plurality of three-dimensional images of at least a portion of a human body acquired over a period of time, wherein the plurality of images comprises at least a first image and a second image acquired at different times;
dividing the first image and the second image into a plurality of regions, wherein each of the regions corresponds between the first image and the second image;
assessing a property in a plurality of regions of the body from the first image;
assessing the property in a corresponding region of the body from the second image; and
comparing the property of the regions of the body from the first image to the property of the regions of the body from the second image to select either a region from the first image or a corresponding region from the second image; and
creating a third image of at least a portion of human blood vessels using the selected regions.

12. (canceled)

13. A method of imaging blood vessels in a human body, comprising:
providing a plurality of three-dimensional images of at least a portion of a human body acquired over a period of time to a computer system, wherein the plurality of images comprises at least a first image and a second image acquired at different times;
dividing the first image and the second image into a plurality of regions, wherein each of the regions corresponds between the first image and the second image;
assessing an intensity in a plurality of regions of the body from the first image;
assessing the intensity in a corresponding region of the body from the second image; and
comparing the intensity of the regions of the body from the first image to the intensity of the regions of the body from the second image to select either a region from the first image or a corresponding region from the second image with the greater intensity; and
creating a third image of blood vessels of the body using the selected regions.

14-90. (canceled)

91. The system of claim 11, wherein the first image and the second image comprise at least a portion of a human heart.

92. The system of claim 11, wherein a region comprises one or more voxels.

93. The system of claim 11, wherein a property comprises an intensity of a region.

94. The system of claim 11, wherein comparing the property of the regions comprises using a mathematical operator to compare the regions.

95. The method of claim 13, wherein the first image and the second image comprise at least a portion of a human heart.

96. The method of claim 13, wherein a region comprises one or more voxels.

97. The method of claim 13, wherein the third image at least appears three-dimensional.
